(19) United States
(12) Patent Application Publication
(10) Pub. No.: US 2006/0198805 A1
(43) Pub. Date: Sep. 7, 2006

(54) Cosmetic and Pharmaceutical Masks for Skin Improvement

(75) Inventor: Shyam K. Gupta, Scottsdale, AZ (US)

Correspondence Address: SHYAM K. GUPTA;BIODERM RESEARCH, 5221 E. WINDROSE DRIVE, SCOTTSDALE, AZ, 85254, US

(73) Assignee: BIODERM RESEARCH, Scottsdale, AZ (US)

(21) Appl. No.: 11/164,709

(22) Filed: Dec. 2, 2005

Related U.S. Application Data
(63) Continuation-in-part of application No. 10/249,701, filed on May 1, 2003, now abandoned.

(51) Int. Cl. A61K 8/73

(52) U.S. Cl. 424/70.13

(57) ABSTRACT

A method for skin improvement, comprising the application of a mask composed of two different fluid components, wherein the first component is adapted to be applied to contact skin, and second component is adapted to be applied to contact the first component, and whereby first and second components chemically react with each other to form a new gel-like component that adapts to the shape of skin as a continuous film, and which is removed from skin as a continuous gel-like film upon being pulled away from skin, and which also removes skin wrinkles, skin blemishes, and skin discoloration as the continuous film is pulled away from skin.

This is a continuation-in-part of U.S. patent application Ser. No. 10/249,701, filed May 1, 2003.

U.S. patent application Ser. No. 10/249,701, filed May 1, 2003, by the present inventor discloses certain cosmetic mask compositions suitable for face, hair, and body application. Surprisingly and unexpectedly, this inventor has now found a method of the application of said mask compositions that removes skin wrinkles, corrects blemish, and reduces skin discolorations.

The enhancement of physical appearance occupies greater focus in human life than nearly all other daily life-related concerns combined. There are far more consumer products available for the beautification of human body than for the treatment of human ailments. The improvement of body tone and appearance is a growing, multibillion-dollar industry encompassing cosmetic, nutraceutical, pharmaceutical, and physical therapy disciplines. The consumer attention is focused on newest miracle ingredient in age defying, anti-wrinkle, body firming, varicose reducing, and slimming compositions. Of all such cosmetic beauty concerns, the enhancement of facial appearance occupies the greatest focus and concern for humans, especially for the female human. The selective treatment of face is thus of paramount importance to consumers. There are a variety of cosmetic delivery systems, such as lotions, creams, sprays, splashes, gels, sticks, and such that have been used to provide beneficial ingredients and compositions to facial areas. A cosmetic mask offers one of the most desirable delivery systems for the delivery of skin beneficial ingredients and compositions to facial skin. However, the development of multifunctional cosmetic masks has been very limited in the prior art.

The appearance of face being the most important concern for mankind, a combination of compositions that includes ingredients to reduce excess fat on face, reduce “double chin”, slim neck area, and to also provide antiaging and anti-wrinkle benefits has been of high consumer interest. A mask product that can be applied to face, chin, and neck areas to deliver the fat reducing, slimming, toning, antiaging, and anti-wrinkle benefits specifically on such site-specific locations of human body for their maximum absorption, bioavailability, and benefits has been in high consumer demand. Such a composition is yet unavailable, as a review of prior art literature has established. Additionally, a mask is considered to have an adsorption effect for removing unwanted oils, pollutants, impurities, and dead cells from the stratum corneum. Removal of the mask is believed to assure deep cleansing of the skin, in particular of the horny layer of the epidermis. It also provides a state of hyper-hydration of the epidermis, resulting in an improvement in skin tone and texture. A scientifically designed mask can also provide skin and body beneficial ingredients for topical delivery for imparting their maximum absorption, bioavailability, and efficacy, as have been known in the prior art.

A number of cosmetic mask compositions have been disclosed in the prior art. For example, among the commercially available mask forming products is a clear gel from Revlon Corporation known as “Honey Masque”. The listed contents include water, ethyl alcohol, polyvinyl alcohol/vinyl acetate copolymer, dimethicone copolyol, propylene glycol, PEG-8, honey, oleth-10 phosphate, fragrance, preservative and colors. A product sold by the Procter & Gamble Company under the trademark of “Noxzema Deep Cleansing Mask” is based upon a polyvinyl alcohol film-forming material solubilized in ethanol; other ingredients include sorbitol, PEG-4 steareth-20, PEG-32, PEG-6, preservatives, fragrance, essential oils and colors.

U.S. patent application Ser. No. 20020039976 (Sebillotte-Arnaud, et al.) discloses a rinsable, foaming, cleansing mask composition comprising at least one hydrophobic silica and at least one oxyalkylenated compound.

U.S. patent application Ser. No. 20030044366 (Dole et al.) discloses a cosmetic mask that changes color upon drying. U.S. patent application Ser. No. 20030014096 (Burkhard) discloses a washable facial mask comprising a unitary frontal element having two eye patch regions made from a washable material, such as cloth.

U.S. patent application Ser. No. 20020088043 (Cook) discloses a cosmetic mask that contains a segment of flexible sheet material and a screen element.

U.S. Pat. No. 6,379,702 (Lorenz et al.) discloses a hydrophilic gel mask, which comprises a blend of acid-neutralized chitosan and a poly (N-vinyl lactam). The gel may be formed into a wound packing or cavity dressing where, unlike hydrocolloid dressings, it is able to absorb exudates without losing its gel structure. It can also be utilized as a drug carrier for transdermal devices and for use in dry skin masks to deliver moisturizers to the skin.

U.S. Pat. No. 6,296,840 (Rodan) discloses a colored facial masque that is formed from a dry powder masque component and an activator liquid component. It is mixed prior to use.

U.S. Pat. No. 5,747,022 (Slavtcheff) discloses a composition for forming a peelable skin mask. It is based on a combination of polyvinyl alcohol and a hydrophobically modified acrylate or methacrylate polymer.

U.S. Pat. No. 6,199,560 (North et al.) discloses a prosthetic cosmetic mask that includes a handle carrying a pad adjacent one end. The pad is formed of a plurality of leaves with a distal edge of each leaf being anatomically contoured with a concave zone to facilitate lateral placement against one's face beneath an eye, the lid of which is to receive makeup. This mask is thus a mechanical device to hold cosmetic ingredients in place on the face.

U.S. Pat. No. 5,893,872 (LaFuente) discloses a prosthetic cosmetic mask having a specially sculpted body contact surface adapted to treat a subject's distinctive bodily characteristics. It is formed by first forming a negative impression of the subject's targeted body area, filling the impression with a hardening material, the hardening material being in the form of a mold reflecting a positive image of the body area. This mask is thus a mechanical device to hold cosmetic compositions in place on the face and other body parts. It does not disclose any chemical compositions that may benefit the appearance of face.

U.S. Pat. No. 5,720,949 (Davis) discloses a cosmetic mask product for application to the skin of the consumer, in particular, to the face of the consumer. The cosmetic mask product comprises a first composition containing an effervescent agent in a cosmetically suitable vehicle and a second composition containing an acid component in a cosmetically suitable vehicle, the first and second compositions being sequentially applied to the skin of the consumer in any convenient order of application. Upon application of the last of the two compositions, a foaming action occurs as a result of gas being liberated by virtue of the reaction between the effervescent agent and the acid component, with concomitant release of heat of reaction. After a time the residue is removed from the skin of the user by means of a scraper or cloth.

U.S. Pat. No. 5,71 6,599 (Golz et al.) discloses a cosmetic preparation containing kaolin that may be employed as a mask, lotion, gel or cream with a non-sticky, non-plasticized consistency and with a content of white kaolin with a high proportion of kaolinite and spherical inorganic particles. This preparation with an inhibitive effect with regard to inflammation permits high kaolin contents to be used in masks and gels. A number of similar masks (called “Mudpacks”), which are based on various clays, are also well known in the prior art.

U.S. Pat. No. 5,599,546 (Klein) discloses another “mudpack” mask composition that includes an acidic material selected from the group consisting of alpha-hydroxy acids, carboxylic acids, halo carboxylic acids, dicarboxylic acids, and combinations thereof, limonene-based oil, an absorbent carrier, and water. The compositions are applied to the skin as a facial mask and allowed to dry. Such “mud-pack” mask compositions are decreasing in their popularity.

U.S. Pat. No. 4,640,932 (Fong et al) describes a facial mask based on inorganic thickening agents, absorbent powders and/or organic gelling agents. Suitable as gelling agents are gelatin, starch, cellulose gums, guar gum, alginates and polyvinyl alcohols. Benzoyl peroxide is present as an active to control or at least mitigate acne vulgaris. Moreover, this product form is different, as it is intended to be immediately washed off.

U.S. Pat. No. 5,139,771 (Gerstein) discloses a rinse-away face masque composition consisting of 1-70% of maltodextrin, about 0.1-15% of a seaweed derivative selected from the group consisting of salts or esters of alginic acid, carrageenan, and agar, and about 20-95% water. It does not contain any facially beneficial compositions for antiaging and slimming combinations, for example.

U.S. Pat. No. 5,026,552 (Gueret et al) discloses a mask formed from a mesh of woven fabric and a hydratable gel confined within holes of the mesh. Since the gel is confined, the mask can be pulled off all in one piece thereby performing a skin sloughing treatment.

U.S. Pat. No. 5,194,253 (Garrido et al) describes a method of forming a cosmetic treatment mask based on at least one hydrophilic film-forming polymer, ammonium hyaluronic acid, mineral or organic salt of deoxyribonucleic acid and water.

U.S. Pat. No. 4,014,995 (Juliano et al.) discloses cosmetic lotion, cream, and mask compositions based on oat flour. This disclosure is specific to oat flour and not applicable to compositions that contain several synergistically combined skin beneficial ingredients.

U.S. Pat. No. 5,158,772 (Davis) discloses topical compositions, including a mask, for application to the skin comprising a topically active agent which may be a cosmetic agent or a therapeutic agent, and a small, but carrier effective amount of a microbial polysaccharide polymer gel which is a beta-1,3 glucan-type polysaccharide.

U.S. Pat. No. 5,690,945 (Bui Bertrand) discloses gelled cosmetic composition for use as a cleansing mask, said composition comprising an aqueous gel and, as principal cleansing agent, graded spheroid polyamide particles that are dispersed in said aqueous gel for skin exfoliation.

Chinese Patent CN1052426 (Youan) discloses a cosmetic film mask made of pollen corpuscles, polyvinyl acetate and polymethacrylate, polyvinyl alcohol, and alginic sodium, polyethylene glycol and glycerol, and a surfactant such as sorbitol monolaurate.

Rumanian Patents RU RU2185147 and RU2184530 (Mikhajlova) disclose cosmetic masks with oat powder (oat flour), baking yeast, and wheat germ flakes.

European Patent EP1186291 (Potin et al.) discloses a cosmetic mask composition comprising water, polyvinyl alcohol, at least one copolymer of vinyl pyrrolidone and at least one oil. It is a cleansing mask.

Canadian Patent CA2202735 discloses a mask product comprising first and second compositions for sequential application to the face of a consumer, one of said composition containing an effervescent agent and the other of said composition containing an acid component.

U.S. patent application Ser. No. 20020025334 (Smith) discloses a composition for application to human or animal skin comprising first and second fluid components, wherein the first component is adapted to be applied to contact the skin, and the second component is adapted to be applied to contact the first component, whereby first and second components in combination are adapted to provide a bilayer comprising a therapeutic layer to contact the skin and a (semi) solid barrier layer, wherein the bilayer is adapted to provide a cohesion which is less than its adhesion, a process for the preparation thereof components thereof and the use thereof in aiding healing, repair and/or protection of human or animal skin. However, Smith compositions, which could be adaptable for a cosmetic mask application, serve to only provide a barrier function or infuse beneficial agents into skin. Smith compositions do not come out as a single gel-like piece in their cosmetic appeal, or remove wrinkles (i.e. not deposit any agents on skin but remove wrinkles from skin), or improve skin discoloration by removing discolored skin, or remove skin blemish. Thus, none of the benefits claimed in present invention are anticipated from Smith teachings. In the present invention, the method of mask application itself provides claimed benefits, and not the agents or ingredients that it infuses into skin or delivers to skin that provide such benefits as claimed in Smith compositions.

The present invention removes skin discoloration or pigmentation commonly called age-spots. U.S. patent application Ser. No. 20010053350 (Chevalier et al.) also discloses similar benefits. However, Chevalier et al. utilize additional agents, such as N-ethyloxycarbonyl-4-para-aminophenol and at least one depigmenting agent selected from arbutin and its derivatives and/or ellagic acid and its derivatives, and its use in a method for depigmenting and/or lightening skin.

In cosmetic mask formulations, both the visual appeal and delivery of high performance skin, body, and hair care ingredients and compositions are important. The compositions from various seas and oceans, that includes the plant parts and their extracts, such as Algae extract, Spirulina, Fucoidan, Laminaria, Corollina, Codium, Ulva lactata, Plankton, Hypnea, Rhodophycea, Ceramium, Fucus, Enteromorpha, Chondrus, Maris, Macrocystis, Gelidium, Pelvetia, Crithmum, Undaria, Ascophyllum, Alaria, Palmaria, Porphyra, Sargassum, Eisenia, Chlorella, and the plants from hydrothermal vents that includes Thermus, Alteromonas, Anacystis, and such, are currently popular antiaging, skin smoothing, and UV-protective ingredients in cosmetic products. The seaweeds are also a very popular item from nutraceutical point of view.

It would thus be highly desirable if a cosmetic and pharmaceutical mask composition that develops the appearance of seaweed after its application, which can also deliver skin, body, and hair beneficial effects, can be invented. Such a composition would have both the visual appeal and the performance attributes desirable from such mask composition(s). Such mask compositions have not been disclosed in the prior art.

### SUMMARY OF INVENTION

The present invention discloses cosmetic and pharmaceutical mask compositions suitable for face, hair, skin, and body applications. These compositions come off from the site of their application essentially in one piece with the appearance, for example, of a piece of seaweed or a continuous film. These mask compositions can also synergistically combine at least one skin, hair, face, or body beneficial cosmetic or drug composition. These mask compositions are suitable for a variety of delivery system methods, such as peel-off mask, leave-in mask, moisturizing mask, exfoliating mask, prosthetic mask, soaking mask, depilatory mask, foaming mask, rinse-off mask, scrubbing mask, rub-off mask, two-phase mask, two-compartment mask, two-phase mask, self-heating (heat-releasing) mask, and such.

The mask compositions of the present invention are made by a novel ion-pair delivery system from certain biopolymer or polymer based films that are further cross-linked with divalent or trivalent metal cations. During the cross-linking process, such divalent and trivalent metal cations may also act as release agents for other face, hair, skin, and body beneficial compositions in their enhanced bioavailable forms.

This invention relates to a method of the application of certain facial, skin, hair, and body mask compositions that come off from the site of their application essentially in one piece with the appearance, for example, of a piece of sea-weed or a continuous gel-like film, and whereupon said application also removes wrinkles and improves skin appearance.

Additionally, such masks are useful as a delivery system for ingredients and compositions for the treatment and regulation of topical disorders of facial area skin, such as skin aging, wrinkles, acne, rosacea, age-spots, striae distensae (stretch marks), pimples, skin infections and lesions, varicose veins, venous insufficiency, skin redness, excess topical fat reduction, cellulite control, and muscle and skin toning benefits, and such.

In a further respect, this invention relates to facial, hair, skin, or body mask compositions that are adaptable for two-phase or two-compartment mask delivery systems, and such.

In a further respect, this invention relates to facial, hair, skin, and body mask compositions that do not require drying before mask removal. In fact, it is beneficial to not let such mask compositions dry out completely after their application, as their being somewhat wet helps their easy removal from the body part(s) where they are applied, and also assists in the penetration of skin, hair, facial, and body beneficial ingredients that such masks may contain. However, this is not a limitation, as the seaweed appearance may still develop even if the mask composition is allowed to completely dry our after its application.

### DETAILED DESCRIPTION

Appearance of face being the most important concern for the mankind, a combination of compositions that includes ingredients to reduce excess fat on face, reduce “double chin”, slim neck area, and also provide anti-aging and anti-wrinkle benefits has been of high consumer interest. The present invention discloses mask compositions that can be applied on face, chin, and neck areas. Surprisingly and unexpectedly, skin wrinkles are also removed as these masks are applied and subsequently removed from their site of application. In addition, skin blemish and skin discolorations are also corrected as these masks are applied and subsequently removed from their site of application.

Facial and body mask compositions of the present invention can be designed in a variety of delivery system forms that includes peel-off mask, leave-in mask, moisturizing mask, exfoliating mask, “fillers” for prosthetic mask, soaking mask, depilatory mask, foaming mask, rinse-off mask, sloughing mask, rub-off mask, two-phase mask, self-heating (heat releasing) mask, and so forth.

A peel-off mask is applied as a liquid film that is thinly spread with fingers on the face or body part. It is allowed to dry for several minutes, then pulled away from face with fingers. It peels-off as a thin plasticized film. It is usually preferred that such masks require a relatively short period of time to dry down to be pulled-off. Such peel-off masks usually provide deep pore cleansing and skin debris removal functions.

A leave-in mask is applied as a liquid, lotion, or cream composition on the face or body part. It is allowed to soak in. It is not rinsed-off or removed by other means. Such masks usually provide skin nutritive and treatment ingredients, such as anti-acne, antibacterial, vitamins, and such that are absorbed with high bioavailability. Various moisturizing masks are generally designed as leave-in mask compositions.

A prosthetic mask is a physical device, such as molded plastic or plaster, that is filled or loaded with a liquid, lotion, cream, paste, or powder “filler” composition and applied to face or body part. It is then left in place for a prescribed period of time. Prosthetic device is then removed and face or body parts rinsed-off.

Exfoliating mask compositions can contain a chemical exfoliator or a physical exfoliator. The examples of chemical exfoliator include various hydroxy acids, fruit acids, and enzymes. The examples of physical exfoliators include various crushed nutshells, luffa particles, sand, polyethylene beads, wax beads, seeds, and such.

A rub-off mask is usually supplied as a paste that is applied to face or body parts and allowed to partially soak in. It is then rubbed with fingers or hand to remove mask compositions that have not soaked into the skin. This mask delivery system thus provides a combination of skin nutritive or treatment and mild skin exfoliation functions.

A foaming mask is applied to pre-wetted face or body part as a liquid or paste, and then rubbed gently with fingers to generate foam. After a few minutes the foam is rinsed off. Such masks thus provide a cleansing and mild exfoliating function. Since the contact time with skin is relatively short, such masks generally do not provide adequate nutritive or treatment benefits.

A soaking mask is a liquid or thin lotion composition that is first soaked onto a piece of fabric or paper. Such fabric or paper pieces may be pre-cut to a shape, such as in the form of a face with holes for nose, lips, and eyes. After soaking, such pieces of fabric or paper are placed on the face and allowed to soak in for several minutes. The fabric or paper piece is then removed. The face may or may not require rinsing at this stage, depending on the composition that was soaked onto the fabric or paper.

Depilatory mask is applied to face or body part for the removal of excess or unwanted hair.

A sloughing mask is usually a combination of foaming mask and exfoliating mask compositions to provide dual, combination benefit.

A self-heating mask releases heat upon application to skin. This is because such masks release heat of hydration when they absorb water from skin surface. The water from skin surface can be from skin that was pre-wetted prior to mask application, or from evaporation of water from skin surface due to natural perspiration.

A two-phase mask is composed of two components, a powder or paste and a thin liquid or activator fluid. These two components are packaged separately. The two components are mixed immediately preceding their application. Another example of a two-phase mask is compositions that are packaged in a two-compartment packaging forms. Such dual-chamber delivery systems, such as U.S. Pat. No. 6,462,025 (Vishnupad), and U.S. Pat. No. 6,448,233 (LaFevre et al.) have been disclosed.

The mask compositions of the present invention are based on ion-pair delivery system. It has been known for some time that some biopolymers and polymers, for example alginic acid and its various monovalent cationic or ammonium salts undergo cross-linking process when reacted with a divalent or trivalent metal cation. This process has been used extensively in various microcapsule compositions, such as those disclosed in U.S. Pat. No. 6,534,091 (Garces et al.), U.S. Pat. No. 6,506,368 (Lages et al.), U.S. Pat. No. 6,497,902 (Ma), and U.S. Patent Applications 20030072803 (Goldenberg et al.), 20030071380 (Wang et al.), 20030064133 (Blatt et al.), 20030055211 (Roberts), 20030021832 (Scherr), and 20020115985 (Larson et al.), for example. However, the application of the cross-linking of algin or its derivatives, such as sodium alginate, in cosmetic and pharmaceutical mask compositions that can be formed and subsequently released as a large continuous, gel-like or rubbery film that also removes wrinkles from skin, or treat skin blemish, or correct skin discoloration during such application has not been disclosed in the prior art.

The method of the application of the mask compositions of the present invention requires the treatment of a film of a cross-linkable biopolymer or polymer with a divalent or trivalent metal cation. In this process, the biopolymer is usually present as a derivative of a monovalent cation or amine salt. This polymer is incorporated in a mask composition of first ion-pair. In Examples section of the present invention, this composition is designated as Part I (Mask Composition). The mask composition of Part I is first applied as a film or layer with fingers on the face or body. This film appears like a paste after its application on face or body. It does not have the appearance of a continuous sheet, or seaweed like, or a gelatinous (jelly fish like), or rubbery appearance at this stage. The cross-linking composition, which is designated as Part II, herein called Ion-Pair Activator Composition, is then applied as a second layer over the first layer of Part I. Upon the ion-pair exchange reaction of the ion-pair composition of the Part I with the ion-pair composition of Part II, i.e. with the divalent or trivalent metal cation, a cross-linking reaction occurs with the release of the monovalent metal cation from composition of Part I, as shown in Scheme 1 for the reaction of sodium alginate with calcium ascorbate as an example. Calcium alginate, produced by the ion-pair exchange reaction between sodium alginate and calcium ascorbate, undergoes cross-linking to form a gel-like continuous sheet of calcium alginate, which appears like a single piece of sheet or like a piece of sea-weed, especially if a green colorant or sea-weed powder is also present in the same composition to provide such desirable visual effects. Calcium ascorbate, which is less bioavailable, is converted into sodium ascorbate, which is more highly bioavailable, during this process, as shown in Scheme 1. Upon the removal of this gel-like continuous sheet from skin by pulling with fingers the wrinkles are also removed and skin is improved.
Na Alginate (not cross-linked)+Ca Ascorbate (less bioavailable)=Ca Alginate (cross-linked)+Na Ascorbate (more bioavailable) [Scheme 1]

However, if the release of a skin or body beneficial composition is not desired, the ion-pair cross-linking process can still make the mask, as shown in Scheme 2.
Na Alginate (not cross-linked)+CaCl2=Ca Alginate (cross-linked)+NaCl [Scheme 2]

Also, as can be noted from Scheme 1, calcium ascorbate is converted into sodium ascorbate in the same ion-pair exchange process. It is well known in the prior art that sodium ascorbate is more bioavailable and is more easily absorbed into the skin than calcium ascorbate. Thus, the ion-pair exchange process permits both the formation of calcium alginate, which appears as a continuous sheet of cross-linked sheet (that may look like a piece of sea-weed, if colored green), and also more bioavailable form of ascorbic acid.

For marketing reasons, and also for consumer convenience, it may be advantageous to produce such ion-pair delivery system mask compositions in two parts. Part I can constitute the main body of mask composition in uncross-linked form. It can also contain other beneficial ingredients and compositions. Part II of this mask composition can be called the “activator”, which contains divalent or trivalent metal cations for the cross-linking of mask composition in Part I. The Part II mask composition can additionally contain other beneficial ingredients. This two-part composition provides an additional advantage in that any beneficial ingredients or compositions that are not stable in Part I can be added in Part II, and vice versa. Part II of the mask composition can also be made in an essentially anhydrous form, if so desired, to provide stability to any ingredients or compositions that are unstable in a hydrous medium. For example, some of the compositions that contain Vitamin C, such as Ester C, are unstable in hydrous media. However, since Ester C also contains Calcium ascorbate, which is a good cross-linking agent for Part I of masks compositions of the present invention. The anhydrous medium of Part II can also be used for self-heating mask compositions as well, as further disclosed in the Examples section of this invention.

### EXAMPLES

The following examples are presented to illustrate presently preferred practice thereof. As illustrations they are not intended to limit the scope of the invention. All quantities are in weight %. Most of these compositions are in two-parts. Part I is usually the mask base. Part II is usually the activator composition that causes the cross-linking of the mask composition.

### Example 1

### Wrinkle Removing Seaweed Facial Mask

Part I. Mask Composition (1) Deionized water 85.5 (2) Sodium Ascorbyl Phosphate 0.5 (3) Spirulina algae powder 1.5 (4) Sodium alginate 2.0 (5) Glycerin 10.0 (6) Preservatives 0.5 Procedure. Mix all ingredients with a high-speed mixer. A green gel is obtained. Part II. Ion-Pair Cross-linking Activator Composition. (1) Deionized water 87.6 (2) Preservative 0.5 (3) Hydroxyethyl cellulose 1.5 (4) Calcium chloride solution (50%) 10.0 (5) Laminaria Extract 0.1 (6) Hydrolyzed Rhodophycea 0.1 (7) Codium Tomentosum extract 0.1 (8) Enteromorpha Compressa extract (0.1). Procedure. Mix (1) to (3) to a thin gel. Add (4) to (8) and mix. A clear light gel is obtained.

### Example 2

### Application of Mask Compositions of Example 1 for Wrinkle Removal

Apply Part I on face or body as a thick film, about the thickness of a penny coin (about 1.0 mm thick). Let it absorb into the facial skin for 10 to 15 minutes. Apply a thick coating of Part II over Part I film. After five to 10 minutes rub the facial skin with fingers. The mask separates from skin as a single piece of gel-like material that appears like a piece of seaweed, also removing wrinkles from skin as it is pulled away from skin.

### Example 3

### Psyllium Seaweed Facial Mask

Part I. Mask Composition. (1) Psyllium husk powder 2.0 (2) PEG-6 30.0 (3) Phaseolamine 1.0 (4) Soybean Fibers 5.0 (5) Oat Protein 5.0 (6) Carnosine 0.5 (7) Water 54.5 (8) Algin 1.5 (9) Preservatives 0.5 Procedure. Mix 1 to 6 to a paste. Mix separately 7 to 9. Add to main batch and mix to a paste. A thin dough-like light brown product is obtained. Part II. Ion-Pair Cross-linking Activator Composition. (1) Deionized water 91.5 (2) Preservative 0.5 (3) Hydroxyethyl cellulose 1.0 (4) Calcium Ascorbate 2.0 (5) Calcium Hydroxycitrate 5.0 Procedure. Mix (1) to (3) to a light gel. Add all other ingredients and mix. A clear light gel is obtained.

### Example 4

### Application of Mask Compositions of Example 3

Apply Part I on face as a thick film, about the thickness of a penny coin (about 1.0 mm thick). Let it absorb into the facial skin for 10 to 15 minutes. Apply a thick coating of part II over Part I film. After five to 10 minutes rub the facial skin with fingers. The mask separates out as a single piece of material that appears like a piece of seaweed, and it improves skin discoloration as it is pulled away from skin.

### Example 5

### A Method for Seaweed Body Mask Application with Skin Lightening Effect

Part I. Mask Composition. (1) Deionized Water 43.9992 (2) Phenoxyethanol 0.7 (3) Methyl Paraben 0.2 (4) Canadian Willow herb 1.1 (5) Chamomile Extract 1.0 (6) Ascorbyl glucosamine 0.1 (7) Micromerol 0.0001 (8) Helioxine 0.0001 (9) Chlorelline 0.0001 (10) Melarrest-L 0.0001 (11) Green Tea Extract 0.0001 (12) Grapeseed Extract 0.0001 (13) Vitamin E Acetate 0.0001 (14) Lipoic Acid 0.0001 (15) Glycerin 50.0 (16) Sodium alginate 2.5 (17) Aloe Vera 0.2 (18) Fragrance 0.2 Procedure: All ingredients are mixed to form a clear gel-like composition. Part II. Ion-Pair Cross-linking Activator Composition (with High Vitamin C content). (1) Glycerin 84.5 (2) Preservative 0.5 (3) Xanthan Gum 1.0 (4) Calcium Ascorbate (Ester C) 10.0 (5) Calcium chloride (50%) 4.0 Procedure. Mix (1) to (3) to a light gel. Add all other ingredients and mix. A clear light yellow gel is obtained. Part I and Part II are applied to skin by the method described herein.

### Example 6

### A Face Mask Composition with Heat-releasing and Wrinkles Reduction Effect

Part I. Mask Composition. (1) Algin 2.5 (2) Sodium Lactate 5.0 (3) Glycerin 18.0 (4) Water 71.3 (5) Hydroxycitric acid, K salt 2.0 (6) Niacinamide 0.5 (7) Glutathione 0.2 (8) Preservatives 0.5 Procedure: Mix 1 to 3 to a paste. Mix 4 to 8 separately to a clear solution. Add this to main batch and mix. A clear gel product is obtained. Part II. Ion-Pair Cross-linking Activator Composition with Heat-releasing Effect. (1) Calcium chloride (Anhydrous) 10.0 (2) Glycerin 52.0 (3) Sodium Potassium Aluminosilicate (Zeolite, anhydrous A3) 30.0 (4) Xanthan Gum 1.0 (5) Spirulina powder 5.0 (6) Dead Sea salt 2.0. Procedure: All ingredients are mixed in a dry atmosphere. A green paste is obtained. Calcium sulfate (anhydrous) or Magnesium sulfate (anhydrous) may be substituted for Calcium Chloride in this example with similar results. Part I and Part II are applied to skin by the method described herein.

### Example 7

### Application of Mask Compositions of Example 6 with Heat-releasing Effect

Apply Part I on face as a thick film, about the thickness of a penny coin (about 1.0 mm thick). Let it absorb into the facial skin for 10 to 15 minutes. Apply a thick coating of Part II over Part I film. Heat release is immediately felt. After five to 10 minutes rub the facial skin with fingers. The mask separates out as a single piece of gel-like material that appears like a piece of seaweed, which removes wrinkles as it is pulled away from skin.

What is claimed is:

1. A method of cosmetic mask application comprising a combination of two different fluid components, wherein the first component is adapted to be applied to skin, and the second component is adapted to be applied to contact the first component, and whereby first and second components chemically react with each other to form a gel-like component that adapts to the shape of skin as a continuous film, and which is removed from skin as a gel-like continuous film upon being pulled away from skin.

2. A method for skin wrinkles reduction, comprising the application of a mask composed of two different fluid components, wherein the first component is adapted to be applied to contact skin, and second component is adapted to be applied to contact the first component, and whereby first and second components chemically react with each other to form a new gel-like component that adapts to the shape of skin as a continuous film, and which is removed from skin as a gel-like continuous film upon pulling away from skin, and which also removes skin wrinkles as the said film is pulled away from skin.

3. A method according to claim 1, wherein the first fluid component is comprised of algin and water.

4. A method according to claim 1, wherein the first fluid component is comprised of algin and water, and wherein algin is from 0.5 to 15.0 by weight percent.

5. A method according to claim 1, wherein the first fluid component is comprised of sodium alginate and water.

6. A method according to claim 1, wherein the first fluid component is comprised of sodium alginate and water, and wherein sodium alginate is from 0.5 to 25.0 by weight percent.

7. A method according to claim 1, wherein the second fluid component is comprised of calcium chloride and water.

8. A method according to claim 1, wherein the second fluid component is comprised of calcium chloride, and wherein calcium chloride is from 0.5 to 50% by weight percent.

9. A method according to claim 1, wherein the second fluid component is comprised of calcium sulfate and water.

10. A method according to claim 1, wherein the second fluid component is comprised of calcium sulfate, and wherein calcium sulfate is from 0.5 to 50% by weight percent.

11. A method according to claim 1, wherein mask composition also includes skin cleansers, cationic surfactants, anionic surfactants, non-ionic surfactants, amphoteric surfactants, zwitterionic surfactants, skin and hair conditioning agents, vitamins, hormones, minerals, plant extracts, anti-inflammatory agents, concentrates of plant extracts, emollients, moisturizers, skin protectants, humectants, silicones, skin soothing ingredients, analgesics, skin penetration enhancers, solubilizers, moisturizers, emollients, anesthetics, colorants, perfumes, preservatives, seeds, broken seed nut shells, silica, clays, beads, luffa particles, polyethylene balls, mica, pH adjusters, processing aids, and combinations thereof.

12. A method according to claim 2, wherein the first fluid component is comprised of algin and water.

13. A method according to claim 2, wherein the first fluid component is comprised of algin and water, and wherein algin is from 0.5 to 15.0 by weight percent.

14. A method according to claim 2, wherein the first fluid component is comprised of sodium alginate and water.

15. A method according to claim 2, wherein the first fluid component is comprised of sodium alginate and water, and wherein sodium alginate is from 0.5 to 25.0 by weight percent.

16. A method according to claim 2, wherein the second fluid component is comprised of calcium chloride and water, and wherein calcium chloride is from 0.5 to 50% by weight percent.

17. A method according to claim 2, wherein the second fluid component is comprised of calcium sulfate and water, and wherein calcium sulfate is from 0.5 to 50% by weight percent.

18. A method according to claim 2, wherein mask composition also includes skin cleansers, cationic surfactants, anionic surfactants, non-ionic surfactants, amphoteric surfactants, zwitterionic surfactants, skin and hair conditioning agents, vitamins, hormones, minerals, plant extracts, anti-inflammatory agents, concentrates of plant extracts, emollients, moisturizers, skin protectants, humectants, silicones, skin soothing ingredients, analgesics, skin penetration enhancers, solubilizers, moisturizers, emollients, anesthetics, colorants, perfumes, preservatives, seeds, broken seed nut shells, silica, clays, beads, luffa particles, polyethylene balls, mica, pH adjusters, processing aids, and combinations thereof.

19. A method for skin discoloration reduction, comprising the application of a mask composed of two different fluid components, wherein the first component is adapted to be applied to contact skin, and second component is adapted to be applied to contact the first component, and whereby first and second components chemically react with each other to form a new gel-like component that adapts to the shape of skin as a continuous film, and which is removed from skin as a gel-like continuous film upon pulling away from skin, and which also removes skin discoloration as the said film is pulled away from skin.
